(19) United States
(12) Patent Application Publication
(10) Pub. No.: US 2009/0156947 A1
(43) Pub. Date: Jun. 18, 2009

(54) Knowledgebased image informatics system and method

(76) Inventors: James B. Seward, Rochester, MN (US); Bijoy K. Khandheria, Fountain Hills, AZ (US); Carl T. George, Minneapolis, MN (US); William H. Hansen, Rochester, MN (US)

Correspondence Address: BECK AND TYSVER P.L.L.C., 2900 THOMAS AVENUE SOUTH, SUITE 100, MINNEAPOLIS, MN, 55416, US

(21) Appl. No.: 12/154,412

(22) Filed: May 22, 2008

Related U.S. Application Data
(60) Provisional application No. 60/931,228, filed on May 22, 2007.
(60) Provisional application No. 60/931,239, filed on May 22, 2007.

(51) Int. Cl. A61B 5/00; G06N 5/02; G06Q 50/00

(52) U.S. Cl. 600/508; 706/47; 705/3

(57) ABSTRACT

The present invention is a system and method for analyzing medical images and image-derived data. Before the medical image procedure is accomplished, the present invention analyzes and reports on the appropriateness of the proposed procedure based upon known client data. After image and image-derived data is received from an image acquisition device, the present invention validates the data against data validation rules. Once the data is validated, it is analyzed against a knowledgebase to create a proposed diagnosis based on the validated data. The knowledgebase is preferably embodied in a binary cascade system.

### CLAIM OF PRIORITY

This application claims the benefit of U.S. Provisional Application No. 60/931,228, filed May 22, 2007, and U.S. Provisional Application No. 60/931,239, filed May 22, 2007.

### FIELD OF THE INVENTION

The present invention relates to the automated analysis of medial imaging system data using a robust clinical knowledgebase. More particularly, the present invention relates to the application of appropriateness testing, intelligent data validation, as well as the automatic generation of a suggested diagnosis from the testing data.

### BACKGROUND OF THE INVENTION

### Terminology

### Data, Metadata, Knowledge, Understanding

In the present disclosure, it is useful to distinguish between data, metadata, and knowledge. Data are numbers or other identifiers derived from observation, calculation, or experiment (e.g., data acquired from an imaging device). Information is data in context—a collection of data and associated explanations, interpretation and other material concerning a particular object, event, or process (e.g., the human interpretation of data importance or relevance). Metadata is data about data, such as data that describes the context in which information was obtained or is used (e.g., descriptive summaries and high-level categorization of data).

The concepts of knowledge and understanding are worth noting because these terms often used interchangeably. Understanding is the use of metadata and information to make logical choices (i.e., the human capacity to render experience intelligible by relating specific knowledge to broad concepts). Knowledge is a combination of metadata and an awareness of the context in which the metadata can be successfully applied and is commonly used in the artificial intelligence community to express how to use information and metadata.

### Informatics and Information Science

Informatics includes the general science of information, the practice of information processing and the engineering of information systems. Informatics studies the structure, behavior, and interactions of natural and artificial systems that store, process and communicate information. Health or medical informatics is the intersection of information science, medicine, and health care. It deals with the resources, devices, and methods required to optimize the acquisition, storage, retrieval, and use of information in health and biomedicine. Information science is an interdisciplinary science primarily concerned with the collection, classification, manipulation, reporting, storage, retrieval, and dissemination of information. Information science and informatics are very similar, with information science generally being considered a branch of computer science and informatics is more closely related to the cognitive and social sciences. In the present disclosure, the term informatics can be treated as synonymous with information science.

### Knowledgebase

Accumulated knowledge, as applied to artificial intelligence algorithms, is commonly referred to as a knowledgebase. Each knowledgebase is unique to the expert or experts from which it emanates. In general, a knowledgebase is a centralized repository for information. In the context of information technology, a knowledgebase is a machine-readable resource for the dissemination of information. An ideal knowledgebase will optimize information collection, organization, and retrieval. The current disclosure deals with the importance of expert knowledgebases—containing data, information, metadata and ultimately knowledge and understanding—as an to aid to more correctly categorize individual medical problems and assist in determining disease occurrence.

### Medical Image Systems

The clinical practice of medicine is permeated by increasingly sophisticated and complex imaging technologies that have proved extremely useful in diagnosing medical conditions of patients. Medical images are typically produced by passing energy through the body and receiving the energy at an imaging device. For instance, echo/Doppler ultrasound images are generated by reflected ultrasound energy off tissues in the body. Other widespread energy sources for medical imaging include magnetic resonance or MR (which uses magnetic energy), CT (X-ray energy), and PET (gamma ray emission).

Cardiovascular imagery in particular is over 30 years old and has advanced tremendously in the ability to image the cardiovascular system and aid in the diagnosis of cardiovascular conditions. Sophisticated echo-based imaging systems, for example, not only produce extremely useful images of the cardiovascular system, but are also able to derive a great deal of useful data relating to the cardiovascular system by analyzing the resulting image. In echocardiography, work in the areas of edge detection and wave analysis has increased the ability of imaging systems to automatically generate this data. For example, such echo-based imaging systems are now capable of automatically measuring of cavity sizes of the heart. Additional image-derived data is not generated automatically by the imaging system, but can be derived from human measurement and analysis of the image or Doppler content. Currently, automated acquisition of image-derived data is principally implemented by the engineering infrastructure within the acquisition device industry (i.e., the magnetic resonance or echo image acquisition devices themselves). While other imaging technologies are also able to derive image related date, echo/Doppler imaging acquires the most data, commonly exceeding 100 or more measures or computations including cardiovascular function, hemodynamics, physiological parameters, and tissue characteristics.

### Shortcomings of Current Medical Image Informatics Systems

### Lack of Automated Interpretation of Data

In the prior art, conventional image informatics systems have focused on obtaining and storing medical images of patients. An example of such a system is set forth in U.S. Pat. No. 6,734,880, issued to Paul Joseph Chang. These systems are able to receive images from image acquisition devices, store the images with patient data, and then present the images to the responsible physician for analysis and diagnosis. Underappreciated is the fact that in many circumstances as much as 70% of the imaging examination is dedicated to the acquisition of data imbedded within the image. Even where prior art systems are able to receive the automatically derived data from the imaging devices, the conventional systems have focused nearly exclusively on simply acquiring and storing the images and related data. The resulting data are then presented to trained individuals (the physician or other technical support) to analyze the collected data in order to convert this data into higher-level information (diagnosis, associations, etc.). This is a labor-intensive process requiring the direct attention of highly skilled individuals. Unfortunately, there is often a shortage of qualified individuals, meaning that image analysis is often delayed. This problem will only increase in the future since image-acquired data is estimated to double every three years.

### Data Inaccuracies

Prior art medical image informatics systems also have significant problems relating to data inaccuracies. Accuracy refers to whether the data correctly records the object or event it represents. Data quality problems have always persisted in databases. In the past, data quality of operational applications tended to reach a point that was acceptable for specific institutional applications. The important data was fixed or processes changed to ensure accuracy and the less important data was not seriously considered for quality improvements. As new uses of data occur, the requirement for accuracy increases. For example, datasets where five percent of the data is invalid due to miss-entry or oversight may be of acceptable quality to the operational applications but unacceptable to a data mining application that attempts to find correlations based on specifics.

Data accuracy has two requirements: it must be the right value and it must represent the value in a consistent form with all other representations of the same value. Invalid data is data that is not the correct value. The most common place for invalid data to enter a medical imaging informatics system is on initial data entry. This means that the wrong value was entered initially. This can result from errors (typos) made by the data entry person, lack of training by entry people, poor data entry form -design or—deliberate mistakes. As more and more data originates from end users instead of through trained data entry staff, the impact of typos, lack of training and poor entry-form design gets larger and larger. Medical databases are increasingly accepting data that is not entered by the most knowledgeable user through Internet transactions and through external data-feeds. Deliberate mistakes result from the person entering data either not knowing the right value and not able or willing to go find it, not wanting you to know the right value or deliberately providing the wrong information. An example of not knowing the right value is where a form requires you to enter a number for a transaction that clearly does not need it. An individual who does not have a correct entry value simply makes up a value. Required fields lead to this type of mistake where the entry person puts in a valid value to get by the requirement of the entry process but does not know what the right value is. Examples of fields that people frequently lie about on data entry are age, weight, common function descriptors, or even names of illnesses.

Another important source of inaccurate data comes from mistakes made in extracting, transforming, and loading data into a secondary data source. The data in the source systems are not wrong. It may be stored in very complex structures, hidden in overloaded fields or represented in unconventional ways. The originating application understands the data structures and data representation rules of the data and has no problem in handling it. The processes built for extracting, transforming and loading data often are built without a thorough knowledge of all the tricks and conventions of the data source. Errors of oversight are made by excluding data that appears to be wrong but is not, or failure to transform values correctly. These types of errors occur all the time and result in data warehouses, decision support applications, and integration applications' seeing data that is much more inaccurate than the sources it draws from. The process turns accurate data into inaccurate data.

The last place where data becomes invalid is when it is taken from the database and incorporated into information objects such as reports, spreadsheets, query results, or portal documents. A failure to understand the true semantics of data and the form of encoding values often leads to inaccurate information generated from accurate data. For example, if a correct value in a database is placed on a report using different units than that intended in the database, originally valid data become invalid on the report.

The other component of accuracy is consistency. For example, a text field containing medical data (such as heart function) that is stated as a percentage may have entries for minus 20%, plus 20% and plus 60%. To someone looking at the individual data entries, these values would not indicate any problem. All entries unambiguously express heart function. Consequently, these values are valid values. However, the values themselves are inconsistent with one another. The reason these inconsistent values are considered inaccurate is that they will provide inaccurate results from general queries that select on a value or that aggregate on the field. Values need to be consistent in representation in order to provide accurate ad hoc query results. The operational application may not care about consistency. However, decision support, data mining and integration applications all require consistency. Examples of inconsistent data that can be found in prior art medical image informatics systems include multiple measures of ejection fraction (%) within the same report that have dissimilar values, or a manually entered value has an inconsistent relationship to an automated data point.

### SUMMARY OF THE INVENTION

Some useful developments have been made in recent years in the computerized analysis of medical images. However, such improvements are principally relegated to data acquisition and not automated computer derived knowledge and understanding as defined by the science of informatics. In other words, automated systems are increasingly capable of analyzing medical images and creating raw data (such as calculating cardiovascular flow rates), but are not capable of creating any useful diagnoses from such data.

The present invention uses a very large clinical, education, and research knowledgebase using a binary decision tool algorithm to create an intelligent informatics system capable of receiving medical imaging data and generating metadata, knowledge, and understanding about the medical condition of the patient. Proposed image studies are compared against the patient data to determine the appropriateness of the study. Acquired medical imaging data is then validated and analyzed against a diagnostic knowledgebase, and then reported automatically as higher-level information (metadata, knowledge and understanding) in the form of a proposed diagnosis. The objective is to increase the efficiency of human data interpretation and thus improve medical care and communication by the application of automation.

A binary algorithm is used to efficiently manipulate input (data) and queries within the implanted knowledgebase to direct various output nodes (diagnoses, correctness, understanding, etc.). The knowledgebase has been accumulated over the past three decades by the principal inventors. A bottom-up binary algorithm processing large amounts of diverse data is capable of producing parallel solutions less expensively than conventional top-down methods. Data (input) are ranked in relation to binary questions (0 and 1; yes and no) within the intermediate layers. The result (knowledge) is a logic statement in accordance with the ranking (weight) and order of the data. The cumulative result is a calibrated expression of disease burden (numbers of positive rankings). Each result connection defines an input-output weight (i.e., a higher level of understanding).

### BRIEF DESCRIPTION OF THE DRAWINGS

FIG. 1 is a flow chart of the basic process of the present invention.

FIG. 2 is a schematic drawing of the system that embodies the present invention.

FIG. 3 is a flow chart of the process for appropriateness testing in the present invention.

FIG. 4 is a flow chart of the process for data validation in the present invention.

### DETAILED DESCRIPTION OF THE INVENTION

### Knowledge and Understanding from Image-Derived Data

The present invention is focused primarily on cardiovascular imaging. In particular, the present invention is currently best applied in the field of echocardiography, due primarily to the wealth of image-derived data that can be obtained by the echo images. In working with the resulting datasets from the echocardiograms, the present invention analyzes both structural data (e.g. dimensions and mass) and functional data (e.g. pressure and flow). Data are typically expressed as raw numbers derived directly from the image (dimension, wall thickness, etc.) or through computations calculated from the raw numbers (mass, volume, ejection fraction, etc.).

As more data is collected from medical images, a huge repository of data is being created. Unfortunately, this data is not generally analyzed by, or even readily available to, the healthcare community. At the same time, there are numerous social and technological impediments to the development of automated systems capable of generating useful knowledge from the available data. Automated knowledge-based data interpretation currently does not exist in the field of sophisticated medical imaging. Current data acquisition is so voluminous, complex, and diverse that an individual investigator cannot adequately draw consistent conclusions (information) and commonly overlooks the coexistence of erroneous data. The present invention knowledgebase overcomes these problems through analysis of the dataset, and is able to alert technicians and physicians of inconsistencies in the data and can even suggest appropriate action or avoidance appropriate to the information derived from the dataset.

The current state of advanced cardiovascular imaging is analogous to the interpretation and diagnosis the electrocardiogram 30 years ago. Diagnostic electrocardiography, which is over 100 years old, records tracings of heart's electrical events. Interpretable data are expressed as voltage, waveforms, electrical duration, and heart rate. As the electrocardiographic technology evolved during the 20th century data gradually evolved to automated information and ultimately to metadata interpretation. Within the past three decades, electrocardiographic interpretation has evolved from a visual over-read to a totally automated data, information and metadata acquisition and interpretation (knowledge and understanding) process. Personal interpretation by the attending physician has been replaced by automated computed diagnosis and simply physician oversight and approval. This interpretive evolution saves time and resources, assures uniformity of interpretation, increases productivity, and improves patient care. This is evolution in data interpretation is looked upon as value added with saved resources being redistributed to more pressing clinical needs and patient care. The present invention is designed to bring this same evolution and advancement to the analysis of echocardiograms and other cardiographic images.

### Method

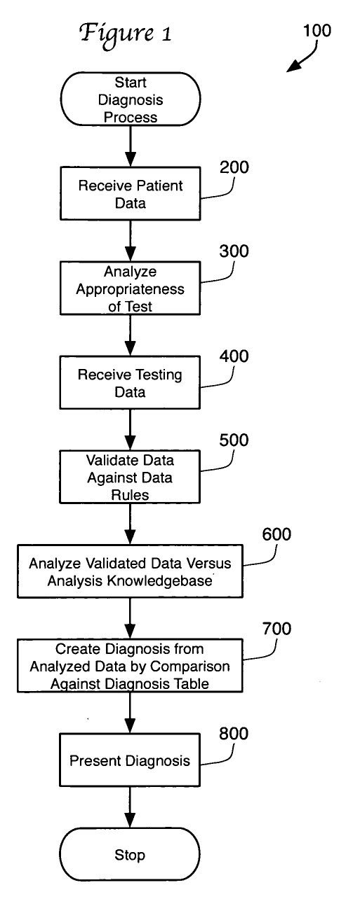

The overall method 100 of the present invention is set forth in FIG. 1. In this method 100, the first step 200 is to receive data about a patient into an automated computerized system. The information received into the system is preferably sufficient to analyze the appropriateness of having a particular imaging procedure given to that patient. This analysis occurs at step 300, and is designed to check and report on appropriate or inappropriate use of the cardiovascular imaging technique for a given clinical scenario as it is represented in the patient data. This analysis is taken from published best practice and appropriateness guidelines that are available to the physicians but are too complicated, detailed, and dynamic to be consistently applied without the automated system of the present invention.

Assuming that the system successfully verifies that a particular imaging technique is appropriate for the presented clinical scenario at step 300, the appropriate imaging technique will be performed and the testing data from imaging device will be received by the system of the present invention. This occurs at step 400. The received data is then subjected to a three-stage analysis in steps 500-700. In step 500, the data if validated against an established validation table to ensure that the data is accurate. In other words, step 500 ensures that the data is complete and not self-contradicting. The validation step 500 helps to prevent numerous false diagnoses.

The validated data is then analyzed against the first stage of the diagnostic knowledgebase at step 600 in order to develop preliminary information about the data. For instance, the analysis at step 600 may identify interpret the data within the image dataset as indicating that the patient has congestive heart failure (CHF). However, this analysis is not complete, as it does not establish a cause for the CHF condition. The further analysis accomplished at step 700, which creates a diagnosis from the information available from the first analysis at step 600. The result at step 700, for instance, may indicate that the patient has congestive heart failure due to a high output state. This is a much more useful diagnosis, which is only possible by converting the data received from the imaging device to the appropriate diagnosis (the knowledge gained from the data) using the knowledgebase of the present invention. Although steps 600 and 700 are shown in FIG. 1 as separate steps in the process 100, it is contemplated and within the scope of the present invention to combine the analysis and knowledgebases used in steps 600 and 700 into a single step and knowledgebase. The suggested diagnosis is then presented to the user of the system in step 800.

### System

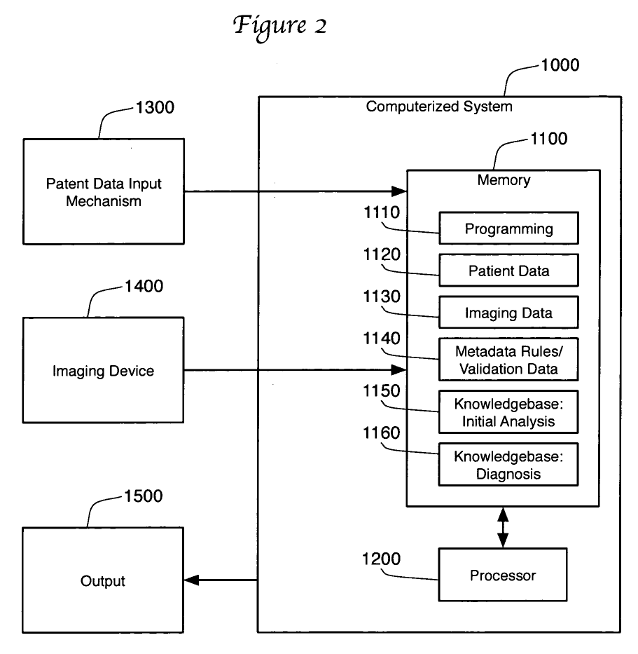

FIG. 2 shows the elements of the system that performs method 100. As seen in that figure, a computerized system 1000 contains a memory 1100 and a processor 1200. The memory 1100 contains software programming 1110 that the processor 1200 uses to operate the computerized system 1000. The processor may be a microcomputer or server processor such as those manufactured Intel. The processor 1200 is also responsible for running the operating system (not shown in FIG. 2) that provides various system capabilities to the computerized system 1000. Any standard operating system could be used by the computerized system 1000, including one of the Microsoft Windows operating systems, Linux, Unix-based systems, or even the Mac OS X operating system provided by Apple Computer. Although FIG. 2 shows the computerized system 1000 in direct communication with the patent data input mechanism 1300, the imaging device 1400, and the output 1500, it is well known that these components could be separated over local or wide area networks, such as the Internet.

The computerized system 1000 works in conjunction with a mechanism 1300 to receive patient data 1120 and an imaging device 1400. The patient data input mechanism 1300 could be as simple as a human interface into the computerized system 1000 to allow manual entry of patient data into the system 1000. Alternatively, the patient data input mechanism 1300 could be data conduit to a second computerized system that already contains the patient data. In this case, the patient data 1120 stored in memory 1100 is retrieved through the data conduit 1300. This data 1120 could be stored in a database format, such as a commercially available relational database management system. Alternatively, the patient data 1120 could be in a proprietary format used by the programming 1110. The programming 1110 and the processor 1200 use the patient data 1120 to perform the appropriateness step 300 described above.

The computerized system 1000 automatically receives images and related data from the imaging device 1300 and stores this information as imaging data 1130. This is described as part of method step 400. The imaging data 1130 contains the actual images created by the imaging device 1300. In addition, the imaging data contains textual and numeric data that is automatically determined by the imaging device 1300 based on the result of the images acquired by the device 1300. For example, in the context of echocardiograms, the imaging device 1400 will be able to provide the system with actual echocardiogram image data as well as data relating to heart dimensions, volume, wall thickness, mass, pressure, flow, and ejection fraction. Additional data may be manually entered into the imaging data 1130 by a technician or physician who can examine and measure the actual image on a computer display or other output 1500 used by the computerized system 100.

The programming 1110 instructs the processor 1200 to compare the imaging data 1130 against the validation knowledgebase established by the validation data 1140 in order to validate the data (step 500 in FIG. 1). The validation data 1140 found in memory 1100 is based upon the knowledge of an expert familiar with the type of data typically received from the imaging device 1400.

After the data is validated, the processor 1200 performs the initial analysis (step 600) on the data using knowledgebase 1150. The result of this analysis is then used as input into the diagnostic knowledgebase 1160 to create an automated diagnostic suggestion for the diagnosing physician.

### Appropriateness

In step 300 of method 100, the system 1000 analyzes the patient data to determine whether the imaging procedure recommended by the physician is appropriate. Imaging studies may have negative consequences, such as poor specificity with a high number of false positives leading to unwarranted further procedures or tests. In addition, imaging studies are expensive and care should be taken to avoid unnecessary or inappropriate imaging studies. A definition of an imaging test's appropriateness must include test performance characteristics for a clinical indication, the potential negative consequences of imaging, and understanding of the implicit impact of cost on clinical decision making, and an explicit understanding of how the test results might lead to care that could improve the patient's chances for better survival or improved health status.

Appropriateness evaluation considers not only the appropriateness of the procedure recommended by a physician, but may also recommend a procedure that the physician has not currently recommended. This helps avoid the under-use of procedures for which the patient would likely benefit as well as the overuse of procedures that may not be necessary or may expose the patient to greater potential harm than benefit. The evaluation is done by comparing patient data against an appropriateness knowledgebase that embodies a comprehensive review of the available evidence and best practices for the management of a clinical condition such as heart failure. This may include the detection or exclusion of disease, as well as risk stratification and the evaluation of therapeutic efficacy. Published guidelines along these lines are found in the appropriate medical journals, such as the Journal of the American College of Cardiology at 2005; 46:1606-1613; at 2006; 48:1475-1497; and at 2005; 46:1587-1605 (each of which hereby are incorporated by reference). The knowledgebase captures recommended guidelines that have been indisputably proven to improve patient outcomes and for which data can be collected that are interpretable, actionable and feasible (See Circulation 2005; 111:1703-1712).

The process for appropriateness testing 300 is shown in FIG. 3. The first step is to receive a recommended imaging procedure, which is generally recommended by an attending physician. This occurs at step 310. This recommendation is then used as the basis for analyzing the patient data 1120 against the validation data knowledgebase 1140 at step 320. If the knowledgebase 1140 indicates that the recommended procedure is appropriate, this appropriateness is confirmed to the user through output 1500 at step 330. If the appropriateness of the procedure cannot be confirmed (i.e., the knowledgebase 1120 indicates that the recommended procedure may be unnecessary or may expose the patient to greater harm than benefit, step 330 will so inform the user. In the preferred embodiment, the portion of the knowledgebase 1120 that found the procedure inappropriate will also provide an output indicating to the user why this finding was made while also citing an authority (such as a journal article) for this determination. The knowledgebase 1120 may also find another imaging procedure to be a better fit given the patient data 1120. If so, step 330 will inform the user of this determination while also citing an authority or rationale for this recommendation.

### Data Validation

Step 500 in the method 100 of the present invention validates the image-derived data against validation rules 1140 before analyzing the data with the diagnosis knowledgebases 1150, 1160. In general, there are two ways to find inaccurate data: re-verify the values or compare the values to rules that are required to be true for the data. Re-verification means that an individual must go back to the originating object or event and see if the data in the database is correct. This approach has many problems: it is very time-consuming and expensive, it may not be possible to examine the original object or event, and the re-verification process may introduce new mistakes.

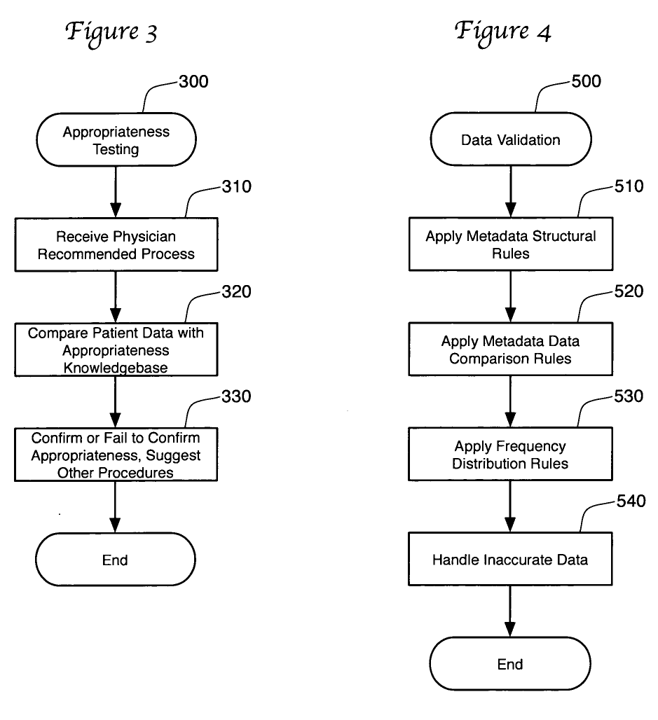

The present system uses the second approach to validate the image-derived data, as shown in the data validation process 500 in FIG. 4. This is accomplished by appropriate definition of the metadata that serves as the validation data 1140. The validation process 500 uses a variety of metadata rules 1140 to help prevent inaccurate data from being used as the basis of knowledgebase analysis in steps 600 and 700. While the use of metadata validation rules 1140 is known in prior art database design, the extent of validation rules in the present invention and the interrelationship between records and other data values is unprecedented in medical image management systems.

Most prior art systems are built by expert engineers with little input from the clinical medial community. Data is assuredly acquired but not managed effectively—data inaccuracies, inconsistencies, and redundancies are common. Because the individuals who develop and maintain these systems are typically not the professionals who interpret or use the data, many important applications lack even a cursory amount of metadata. The developers have little knowledge from which they may assess data accuracy, and the knowledge they do have is often inaccurate and outdated. Nonetheless, the metadata developed by these developers ends up defining what constitutes accurate data in these prior art systems.

### Structural Rules

Metadata rules 1140 provide a definition of what values are valid for each field. For example, it can provide a data type, length restrictions, range of acceptable values, discrete list of acceptable values, rules for entering “not known” or “not applicable” entries and other rules such as uniqueness or consecutiveness. These definitions restrict the values to only those that are valid for the field. These types of metadata rules are known as structural rules, and are generally well known in the prior art. The application of the structural metadata rules to the imaging data 1130 is shown as step 510 in FIG. 4.

### Data Comparison Rules

While structural rules help detect and prevent invalid values, it is possible for a value to be both valid and yet not accurate. For example, if a field named ejection fraction contains a value of −20%, it is invalid since it is not a valid number. If it contains a value of +20%, it is valid. However, to be accurate, the situation it refers to must have “abnormal function” otherwise this value is valid but inaccurate.

To help catch additional inaccurate data, the present invention uses data comparison rules. Data comparison rules are designed to find incompatibilities between two or more values, each of which pass the valid value tests. Examples are:
A normal ejection fraction must ≧50 percent.
However, if the patient has aortic valve regurgitation then a normal ejection fraction must be ≧65 percent.

Data comparison rules must be based on a comprehensive understanding of multiple disease or event scenarios. These rules encode conditions that must exist within a set of objects of the same type or across object types. They embody medical knowledge about how data must compare to other data. This comparison can be between different types of data such as in the above example comparing ejection fraction with the existence of aortic valve regurgitation. The comparison may also take place between the same types of data over time. For example, the present invention recognizes that the ejection fraction for a particular patient must be consistent over time or within a designated time frame. If there are outliers among the data (i.e., ejection fraction values that are out of line with other ejection fraction values obtained for this patient), they can be flagged and queried. Data comparison rules are applied at step 520 in FIG. 4.

### Frequency Distribution Rules

The present invention also defines value frequency distribution rules (step 530 of FIG. 4). These rules help locate cases where one value is represented too frequently or other cases where a value is not represented enough or not at all. These frequency distribution rules are based upon statistical analysis of prior data sets, and can be used to spot inaccurate data entries. For instance, these types of rules will spot situations where a person responsible for data entry frequently uses the default value (or a made up value) in lieu of entering the correct values. These rules are effective because individuals who make up values tend to use the same false value repeatedly.

### Handling Inaccurate Data

Once inaccurate data is identified, the data can be flagged for user evaluation or the system can attempt to correct the inaccuracy. This is done at step 540 of FIG. 4. If the system is flagging data inaccuracies, any new data that fails a data validation rule 1140 will be flagged as inaccurate for further evaluation by the user. As for correcting inaccurate data, there are three established ways to accomplish this: manual re-verification, transformation, and correlation. Manual re-verification is clearly more useful in this context since you are focusing only on the subset of the data where you know inaccurate values exist. If the data source has an excessive amount of wrong data exposed, it may not be practical to use. The problem with manual re-verification is that a user must sort through the original data and determine the source of the error. Although most cases require this individual attention, automated methods of correction would be valuable in order to save the user the inconvenience of manual re-verification.

Transformation is an automated technique usable when a list of wrong values can be easily associated with correct values. These are useful for fixing representation problems and common misspellings. The system contains a list of wrong values that have been found in the past and where the correct value is obvious. In this case, the inaccurate value is automatically replaced with the correct value without user interaction.

Correlation is another automated technique that is used in cases where multiple fields must correlate. If inaccurate data is detected, the system can sometimes determine which value is wrong and substitute the correct value that would make the rule work. This is a very common technique for name and address scrubbing routines. It is very helpful in cases where one of the fields is missing or a single value is misspelled. The problem with correlation and transformation routines is that they are never 100 percent accurate in their corrections. Nonetheless, in most cases the system can determine to a very high probability that the change is correct. If the system makes a wrong guess at the correct values only rarely, then transformation and correlation are useful routines to automatically correct inaccurate data.

### Diagnostic Knowledgebase

At steps 500 and 600 of FIG. 1, the validated data is analyzed by the diagnostic knowledgebases 1150 and 1160 in order to determine a suggested diagnosis. From human experience with sophisticated medical imagery and data acquisition, which has evolved over the past three decades, a large understanding of diagnostic correlations has been accumulated. As a consequence, a large knowledgebase (human understanding) has evolved.

At the most abstract level, a knowledgebase can be considered a human trait and not a litany of written information statements or diagnostic criteria. The present invention uses an artificial intelligence knowledgebase, which incorporates aspects of human understanding based on human understanding and interpretation of a vast number of image-datasets. In the present case, the inventors have accumulated a human knowledgebase applicable for management of sophisticated image data through medical examinations and image interpretation over a period of approximately thirty years. Various technological and time sensitive information issues as well as the complexity and volume of image-derived data has precluded earlier development of an automated medical imaging informatics solution.

### Binary Cascade

To embody this human knowledgebase in an automated system, the present invention utilizes a binary cascade based system. A binary cascade system is one in which resulting information can be accessed by asking a series of yes/no questions concerning the data until an answer node is reached. In effect, the binary cascade systems operate in the form of a binary search algorithm. Information measure is applied to a message that consists of a string of binary possibilities each of which is equally likely (yes or no, zero or one, etc.) at every step along the string. To find the information content of any message the message is translated into binary code and digits (bits) resulting in a string of zeroes and ones. The technique is known, somewhat dismissively, as bit-counting. The technique is to divide the possible answers into two roughly equal batches, over and over again. As long as each question (data point) is designed to differentiate between more or less equally probable alternatives, each answer reveals one bit. For example twenty data points (bits) transformed by the binary cascade will correspond to a single choice among 1,048,576 equally probable alternatives (a number computed by multiplying together twenty factors of two). This technique is capable of substantially compressing large volumes of data into information and metadata, as well as begins to address the human qualities of knowledge and understanding.

An active-testing surveillance system (i.e., sophisticated image device) is the communication system from which data (random input X and an response vector Y, and a channel defined by conditional probabilities P(Y/X)), with the added feature that the channel can be changed or manipulated, either deterministically, programmatically or adaptively in response to the knowledgebase observations Y. This informatic-theoretic inference coincides exactly with the Bayesian view. Given a prior distribution px(x) and the transition probabilities py|x(x|y), the observer updates its state of knowledge of X using Bayes' Theorem. The updating procedure can be repeated in a recursion (i.e., binary algorithm), with X possibly changing between measurements as occurs in tracking problem. Based on the probabilities py|x(x|y) decisions can be made and actions taken, depending on the context. The idea of getting to a desired state of knowledge by asking the right questions (from the knowledgebase) in the right order and context is central to the success. At its core, the problem is one of experimental design, sometimes called preposterior analysis in Bayesian statistics (See J. Berger, Statistical Decision Theory: Foundations, Concepts and Methods, Springer-Verlag, New York, 1980)(B. Carlin and T. Louis, Bayes and Empirical Bayes Methods for data Analysis, Chapman and Hall, London, 1996). The general problem of hierarchical decision tree design for classification has much in common with the active-testing surveillance problem. The binary algorithms used in the present invention can be considered a generalization of sequential detection.

### Bottom-Up Processing

Cognitive systems are organized hierarchically. The most basic systems are located at the bottom of the system (example: data). The more complex systems (knowledge and understanding) are located at the top of the hierarchy. Information can flow both from the bottom of the system to the top of the system (also known a data-driven processing) or from the top of the system to the bottom of the system (also known as conceptually-driven processing). When information flows from lower function to higher function the process is call bottom-up processing. The process disclosed is the use of complex data (bottom) validated against a robust knowledgebase (expert knowledge) and reported as higher-dimensional processes (information, metadata, knowledge and understanding). Lower level data systems categorize and describe incoming information and pass this descriptive information onto higher levels for more complex processing.

As disclosed herein individual parts of the system (data) are specified in detail. The data are then linked together (contracted and validated against a knowledgebase) to form larger components, which are in turn linked until a complete system is formed (i.e., diagnosis based on high-level information). The bottom-up approach emphasizes binary coding and validation, which can begin as soon as the first module has been specified (each binary question has same likelihood of “yes” or “no”). This approach has logical limitations, which can be lessened by continuous refinement and artificial learning. Re-usability of code is one of the main benefits of the bottom-up binary approach. Bottom-up parsing is a strategy for analyzing unknown data relationships that attempt to identify the most fundamental units first and then to infer higher-order information from them. Bottom-up approaches, broadly speaking, are able to produce solutions in parallel much more inexpensively that top-down methods. An important component of this disclosure is the existence of a large robust clinically proven knowledgebase as is present in the present invention.

The many features and advantages of the invention are apparent from the above description. Numerous modifications and variations will readily occur to those skilled in the art. Since such modifications are possible, the invention is not to be limited to the exact construction and operation illustrated and described. Rather, the present invention should be limited only by the following claims.

What is claimed is:

1. A computerized method of analyzing medical cardiographic image-derived data comprising:
a) receiving, at a computerized system, patient data containing data concerning the health and identity of a patient;
b) receiving, at the computerized system, a proposed imaging procedure;
c) analyzing, using a computerized knowledgebase at the computerized system, the proposed imaging procedure in light of the received patient data to determine the appropriateness of the proposed imaging procedure;
d) receiving, at the computerized system, image-derived data;
e) applying, at the computerized system, data validation rules against the image-derived data to determine data accuracy and develop validated data by:
i) applying structural rules to the image-derived data;
ii) applying data comparison rules to the image-derived data; and
iii) applying frequency distribution rules;
f) analyzing, at the computerized system, the validated data against a diagnostic knowledgebase in order to develop higher-level diagnosis information.

2. The computerized method of claim 1, wherein the diagnostic knowledgebase is implemented using a binary cascade.

3. A computerized system for analyzing medical cardiographic image-derived data comprising:
a) means for receiving patient data containing data concerning the health and identity of a patient;
b) means for receiving a proposed imaging procedure;
c) means for analyzing using a computerized knowledgebase the proposed imaging procedure in light of the received patient data to determine the appropriateness of the proposed imaging procedure;
d) means for receiving image-derived data;
e) means for applying data validation rules against the image-derived data to determine data accuracy and develop validated data by:
i) applying structural rules to the image-derived data;
ii) applying data comparison rules to the image-derived data; and
iii) applying frequency distribution rules; and
f) means for analyzing the validated data against a diagnostic knowledgebase in order to develop higher-level diagnosis information.
